(19) United States
(12) Patent Application Publication
(10) Pub. No.: US 2026/0294674 A1
(43) Pub. Date: Oct. 1, 2026

(54) HEAT TREATMENT DEVICE

(71) Applicant: Gerd Mattheiss, Trimmis (CZ)

(72) Inventors: Gerd MATTHEISS, Trimmis (CH); Otto Pecher, Aying bei München (DE); Thomas Zeiger, Vomp (AT)

(21) Appl. No.: 19/119,042

(22) PCT Filed: Oct. 9, 2023
(86) PCT No.: PCT/EP2023/077919

§ 371 Date: Apr. 10, 2026

(30) Foreign Application Priority Data
Oct. 7, 2022 (EP) 22200325.3

(51) Int. Cl. A61F 7/00

(52) U.S. Cl. CPC A61F 7/0053; A61F 7/007; A61F 2007/0077; A61F 2007/0088; A61F 2007/0093; A61F 2007/0095

(57) ABSTRACT

A heat treatment device (100; 220) includes at least one infrared radiator (110, 112) including at least one controllable heat source to emit infrared radiation to a skin area of a user of the heat treatment device (100; 200). At least one heat sensor (140, 142) is arranged to detect a surface temperature at the user's skin area, and a controller (160) is configured to control a heat output of the at least one infrared radiator (110, 112) based on the detected surface temperature at the user's skin area. When operating with a thermal output delivered to the user in the range of 20 to 120 milliwatts per square centimeter at the user's skin area, the infrared radiator (110, 112) has a minimum delay of less than 5 seconds to achieve a difference of at least 20% in the thermal output delivered to the user in the skin area.

The application relates to a heat treatment apparatus according to the preamble of claim 1. The application also relates to a heat cabin and a method.

### TECHNICAL BACKGROUND

Heat treatment apparatuses are known. They are usually used to apply heat in the form of thermal or infrared radiation to the body or specific areas of the user's body. Heat treatment is often used as a therapeutic or therapy-accompanying measure, for example as part of physiotherapy treatments, or generally to promote well-being.

The human body does not have an absolute sense of temperature. Furthermore, in the context of heat treatments, even temperatures that may lead to damage to the skin due to excessive heating are often not perceived as uncomfortably hot by many users. In addition, appropriate temperature control in heat treatment apparatuses is complicated by the fact that the surface temperature in a heat-irradiated skin area is highly dependent on the user's distance from the heat source. In the case of installed heat treatment apparatuses, for example in heat cabins or when using a ceiling or wall mount, this distance often varies depending on the user's height or the irradiated body region. In addition, the surface temperature depends on the skin's ability to absorb and process heat depending on the skin's blood circulation and the heat dissipating capacity of the respective user. The heat dissipating capacity is mainly determined by heat transport from the skin to deeper regions of the body as a result of blood circulation and varies from user to user.

In order to achieve sufficient heating of the skin surface for an intended heat treatment, but at the same time avoid injury to the skin due to excessive and/or prolonged heat radiation, it is known to repeatedly detect a skin temperature in an irradiated skin area during a heat treatment using sensors. The recorded skin temperature is then automatically compared with a reference value, for example a stored upper threshold temperature. If the comparison shows that the detected skin temperature is greater than the upper threshold temperature, the heat output of the heat treatment apparatus is automatically reduced. For example, the heat output is reduced for a certain period of time and/or by a certain amount so that further heating of the skin region is avoided or the overheating of the skin region may subside.

In some such heat treatment apparatuses, it is also known not to reduce or possibly even increase a heat output if the detected skin temperature in the irradiated skin area is below a lower threshold temperature, which is lower than the upper threshold temperature. This allows the heat output to be controlled in accordance with a target temperature. In particular, this makes it possible to keep the skin temperature in the irradiated skin area stable over a longer period of time within a predetermined range around a target temperature intended for the heat treatment, regardless of the user.

Ceramic radiant heaters are used in known heat treatment apparatuses in which a heat output may be controlled as described above on the basis of a detected skin temperature within a range around a target temperature. In some ceramic radiant heaters, an electric heating current is passed through a ceramic heating element. An electrical resistance in the ceramic leads to the conversion of part of the applied heating current into heat, part of which is emitted from the ceramic element in the form of infrared radiation. In other ceramic radiant heaters, a ceramic sheath surrounds a metal heating wire.

The ceramic casing is heated from the inside by the heating wire and emits the heat to the outside. The ceramic casing favors uniform heat radiation over an enlarged radiating surface. To increase the heat conduction between the heating wire and the ceramic casing, in some cases an intermediate space is filled with quartz or so-called lava sand. Ceramic radiant heaters are also characterized by a relatively stable heat radiation behavior, even with fluctuations in the heating current and even with the larger spatial extension of the heating element, as is usually the case with ceramic elements.

The control of the infrared radiator according to DE 10 2014 106497 does not provide for an operating state as claimed.

Based on this, one problem is to provide a heat treatment apparatus with extended application possibilities and/or improved injury safety.

### SUMMARY OF THE DISCLOSURE

The problem is solved by a heat treatment apparatus with the features according to claim 1, a heat cabin according to claim 13 and a method according to claim 15.

Thus, according to one aspect, a heat treatment apparatus comprises at least one infrared radiator comprising at least one controllable heat source and arranged to emit infrared radiation generated by the heat source to a skin area of a user of the heat treatment apparatus. The heat treatment apparatus further comprises at least one heat sensor arranged to detect a surface temperature in the skin area of the user, and a control means adapted to control a heat output of the at least one infrared radiator at least partially based on the detected surface temperature at the skin area of the user. The infrared radiator, in an operating state corresponding to a heat output delivered to the user in the range of 20 to 120 milliwatts per square centimeter at the skin area of the user, has a minimum delay of less than 5 seconds to achieve a difference of at least 20% in the heat output delivered to the user at the skin area.

A heat treatment apparatus according to the disclosure allows an improved determination of a heat reduction rate of the user. The heat reduction rate may be determined in particular by means of brief heating of an area of the user's skin and/or by means of brief variations in a heat output delivered to the user at the skin area. This enables more precise and safer control of the heat output of the infrared radiator during a heat treatment in accordance with a heat reduction rate of the user.

Such a heat treatment apparatus also allows improved adjustment of a heat output delivered to the user as a function of a surface temperature at the skin area of the user that is detected, in particular continuously, in the event of changing external conditions during a heat treatment. These include, for example, body movements of the user that significantly change the distance of the user from the at least one infrared radiator. This reduces the risk of injury due to overheating. In addition, the occurrence of insufficient heat output may be reduced in such cases. This improves the effectiveness of the heat treatment. The heat treatment apparatus also allows an extension of heat treatments that may be performed. In particular, heat treatments according to an intended time-varying profile of the surface temperature at the skin area of the user, which provide for a variation in the heat output delivered to the user in a relatively short period of time, are favored. This includes stimulation treatments. This is also possible without compromising the reliability of heat regulation.

The heat treatment apparatus allows the heat output delivered to the user in the skin area to be varied with a delay that is less than is required to establish a thermodynamic equilibrium between a heat output delivered to the user and a surface temperature in the skin area that changes simultaneously as a result of the variation in heat output. This favors heat stimulation treatments, in particular by briefly increasing and/or decreasing the heat output delivered. It also favors calibration of the heat treatment apparatus according to a user-specific heat dissipating capacity by briefly increasing and/or decreasing the heat output, as described in greater detail below.

The heat treatment apparatus also allows heat treatments to be carried out that provide a constant or slightly variable heat output over a longer period of time. This may include heating applications aimed at heating larger areas of the body, including the user's entire body, in particular when the heat treatment apparatus is used in a heat cabin.

The control means may be configured to control the heat output of the infrared radiator, in particular completely, by controlling a heating current of the infrared radiator. The infrared radiator may be configured to achieve the difference in the heat output delivered to the user in the skin area as a result of a variable heating current. In general, the heat treatment apparatus may be configured to achieve the variation in the heat output delivered to the user by correspondingly varying a radiation intensity generated by the infrared radiator.

The control means and the infrared radiator may be configured to achieve the difference in the heat output emitted to the user at the skin area at least in part by proportionally varying the heating current, i.e. not limited to a binary switching on or off of an identical heating current.

The heat source may comprise a ceramic-free heat-generating element. In addition, the heat source may comprise a controllable halogen lamp, a controllable infrared, IR, light-emitting diode, LED and/or a controllable carbon fiber heating element. In particular, the heat source may comprise a controllable halogen lamp which comprises at least one filament as a heat-generating element. Additionally or alternatively, the heat source may comprise a controllable IR LED comprising at least one semiconductor device as a heat-generating element. Additionally or alternatively, the heat source may comprise a controllable carbon fiber heating element which comprises as heat-generating element at least one carbon fiber to which a heating current may be applied.

The heat source may comprise a halogen lamp without an absorption filter. This prevents a filter of the infrared radiator, for example a ceramic or metal-coated cover glass, from being heated as a result of (partial) absorption of the infrared radiation generated by the infrared radiator and delays a variation in the heat output emitted to the user by the infrared radiator due to the accumulation of heat or the radiation of accumulated heat.

The use of a heat source comprising a halogen lamp is advantageous in many applications due to the relatively broad wavelength spectrum of infrared radiation that may be generated by a halogen lamp. In contrast, IR LEDS generate infrared radiation in narrow wavelength bands due to their function. The use of a heat source comprising a halogen lamp is also advantageous in some of the applications described herein due to a typically lower delay in varying the heat output delivered to the user by the infrared heater compared to carbon fiber heating elements.

The infrared radiator may have a minimum delay of less than 4, or less than 3, or less than 2.5 seconds to achieve a difference of at least 30%, or at least 35%, or at least 40% in the thermal power delivered to the user at the skin area in the operating state corresponding to a thermal power delivered to the user in the range of 20 to 120 milliwatts per square centimeter at the skin area of the user.

Additionally or alternatively, in the operating state corresponding to a heat output delivered to the user in the range of 20 to 120 milliwatts per square centimeter at the skin area of the user, the infrared radiator may have a minimum delay of at least 0.5, in particular at least 1 second to achieve a difference of at least 50%, in particular at least 80% in the heat output delivered to the user in the skin area.

The control means may be adapted to control the heat output at least in part as part of a heat output control based on the detected surface temperature.

The control means may be adapted to control the heat treatment apparatus to determine a user-specific heat dissipating capacity at the skin area of the user. Furthermore, after determining the user-specific heat dissipating capacity, the control means may be configured to carry out the heat output control in accordance with a control span that is determined at least in part by the user-specific heat dissipating capacity. The efficiency of the heat output control may thus be improved.

Additionally or alternatively, the control means may be adapted to carry out the heat output control according to an expected body reaction of the user, which is at least partially determined by the user-specific heat dissipating capacity, after determining the user-specific heat dissipating capacity. This enables more precise and safer control of the heat output of the infrared radiator.

Determining the user-specific heat dissipating capacity may comprise determining an initial surface temperature in the skin area of the user. Further, the determining may include varying a heat output of the infrared radiator so as to effect a difference of at least 20% in the heat output delivered to the user in the skin area of the user within a time period of less than 5 seconds, and determining a temperature profile at the skin area of the user based at least in part on a surface temperature at the skin area of the user, which is detected by means of the heat sensor after the heat output has been varied. The temperature profile thereby indicates a heat dissipating capacity of the user.

Determining the initial surface temperature may comprise heating the skin area of the user by use of infrared radiation generated by means of the infrared radiator to attain the initial surface temperature. Varying the heat output may comprise decreasing the heat output. The temperature profile corresponds to a cooling of the skin area at least in part due to heat dissipation in the user's body.

Alternatively, varying the heat output may comprise increasing the heat output. The temperature profile corresponds to a heating of the skin area which is at least in part determined, in particular delayed, by heat dissipation in the user's body.

The heat output control may have a maximum upper threshold temperature of less than 45 degrees Celsius, or less than 44.5 degrees Celsius, or no more than 44 degrees Celsius. This may ensure that skin damage due to overheating is avoided within the usual duration of heat treatments, for example up to maximum durations of at least 2 hours, or at least 3 hours, or at least 4 hours.

The control means may comprise a memory device. The memory device may be configured to store program code for operating the heat treatment apparatus according to at least one heat treatment program.

The memory device may include program code for operating the heat treatment apparatus according to at least one heat treatment program that provides, during a heat treatment, in at least one time segment of less than 5 seconds, a difference of at least 20% in the heat output delivered to the user at the skin area of the user. The heat treatment program may comprise a program for a heat stimulation treatment.

The heat treatment apparatus may also comprise at least one mirror element, which is configured to redirect infrared radiation emitted by means of the infrared radiator toward the skin area of the user. The heat sensor may be arranged in an area of the mirror element. A detection area of the heat sensor may extend at least in part in a portion of a radiation cone that is determined by the redirected infrared radiation.

The heat treatment apparatus may comprise at least two mirror elements and at least two infrared radiators arranged on different sides with respect to the mirror elements. The at least two mirror elements may be configured as a single-piece element.

The heat source of each of the infrared radiators may comprise a controllable halogen tube. The halogen tubes may be aligned at least substantially parallel to one another. A mirror surface of each of the mirror elements may be aligned at least substantially parallel to a longitudinal extension direction of at least one of the halogen tubes.

The heat treatment apparatus may comprise a plurality of heat sensors arranged to detect a surface temperature in each of different sub-areas of the skin area of the user. The control means may be configured to control the heat output based at least in part on the detected surface temperature in the different sub-areas of the skin area of the user.

The skin area of the user may have an area of at least 400 square centimeters.

The heat treatment apparatus may be configured such that in a volume that extends from the skin area and up to a distance of at least 10 centimeters from at least one point of the skin area parallel to a direction of incidence of the infrared radiation, a location-dependent power density of the infrared radiation at any different points in the volume differs by at most 8%.

According to a further aspect, a heat cabin is presented, more specifically an infrared cabin. The heat cabin comprises a heat treatment apparatus of the type presented herein.

The heat cabin may be configured as a pressure chamber, in particular for the purpose of hyperbaric therapy applications. The heat treatment apparatus may be configured for use during a hyperbaric therapy application.

The above-described heat cabin for hyperbaric therapy applications demonstrates that the disclosed heat treatment apparatus is suitable and may be used in various environments and situations.

According to a further aspect, a method for user-related calibration of a heat treatment apparatus is presented. The heat treatment apparatus is a heat treatment apparatus of the type presented herein. The method comprises the steps of determining an initial surface temperature at the skin area of the user. The method further comprises varying, by use of the control means and the infrared radiator, a heat output of the infrared radiator in such a way that in a period of less than 5 seconds a difference of at least 20% in the heat output delivered to the user at the skin area of the user is attained, detecting, by means of the heat sensor and after varying the heat output, a surface temperature at the skin area of the user, and determining, by means of the control means and based at least in part on the detected surface temperature, a temperature profile at the skin area of the user. The temperature profile indicates a user's heat dissipating capacity. The method further comprises determining, by means of the control means and at least in part based on the user's heat dissipating capacity, a control span for a heat output control of the heat treatment apparatus, and/or storing, by means of a memory device of the control means, at least one user-related characteristic value that indicates the user's heat dissipating capacity.

### BRIEF DESCRIPTION OF THE DRAWINGS

Further features, advantages and objectives of the disclosure will become clear from the drawings and the detailed description. In the drawings:

FIG. 1 shows a heat treatment apparatus according to an example;

FIG. 2 shows a heat cabin according to an example; and

FIG. 3 shows a method for calibrating a heat treatment apparatus according to an example.

### DETAILED DESCRIPTION

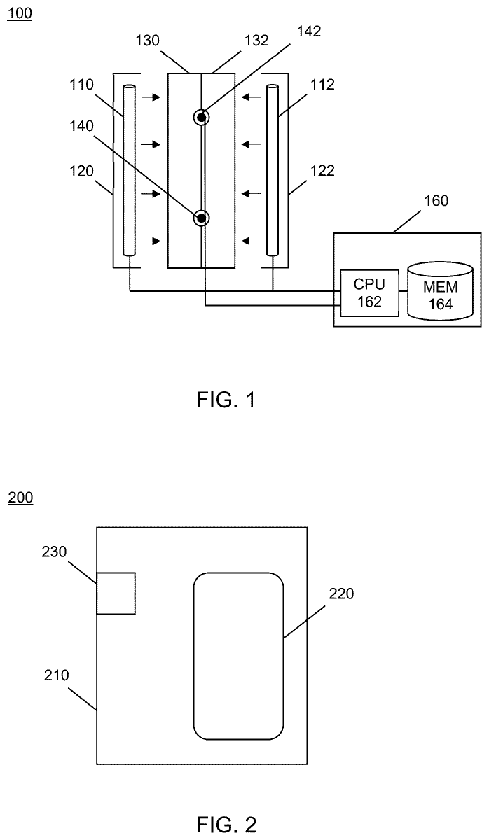

FIG. 1 schematically and exemplarily shows a heat treatment apparatus 100. The heat treatment apparatus 100 comprises several infrared radiators 110, 112.

Each of the infrared radiators 110, 112 is arranged in a housing area 120, 122 of the heat treatment apparatus 100. The heat treatment apparatus 100 further comprises a plurality of mirror elements 130, 132 and a plurality of heat sensors 140, 142 arranged in the region of the mirror elements 130, 132. The heat treatment apparatus 100 further comprises a control means 160 having a processor unit 162 and a memory device 164. The control means 160 is operatively connected to the heat sensors 140, 142 and to the infrared radiators 110, 112.

The infrared radiators 110, 112 are arranged on different sides of the mirror elements 130, 132. As shown schematically by arrows in FIG. 1, each of the infrared radiators 110, 112 is arranged in such a way that infrared radiation generated by means of a heat source of each of the infrared radiators 110, 112 is emitted in the direction of the mirror elements 130, 132.

The mirror elements 130, 132 are arranged in such a way that infrared radiation emitted by each of the infrared radiators 110, 112 strikes a mirror surface of at least one of the mirror elements 130, 132, which is arranged facing the respective infrared radiator 110, 112. The mirror elements 130, 132 are aligned in such a way that infrared radiation coming from a respectively associated infrared radiator 110, 112 and striking at least one of the mirror elements 130, 132 is redirected in the direction of a user of the heat treatment apparatus 100. The user is located in an irradiation area of the heat treatment apparatus 100. In the example of FIG. 1, the irradiation area of the heat treatment apparatus 100 is located above the plane of the drawing. The mirror elements 130, 132 are arranged, for example, in the shape of a roof or in the form of an inverted “V” in relation to the drawing plane. Infrared radiation, which hits the side of one of the mirror elements 130, 132 from the infrared radiators 130, 132, is thus redirected in a direction perpendicular to the drawing plane by means of the mirror elements 130, 132.

The heat sensors 140, 142 are arranged between the mirror elements 130, 132. The mirror elements 130, 132 have recesses for this purpose, for example. This allows the heat sensors 140, 142 to be arranged behind the mirror elements 130, 132. In this way, the heat sensors 140, 142 are shielded from direct infrared radiation which is emitted by the infrared radiators 110, 112 and which could otherwise falsify a measurement result of the heat sensors 140, 142. The heat sensors 140, 142 are aligned in such a way that a detection area of each of the heat sensors 140, 142 at least partially overlaps with the irradiation area of the heat treatment apparatus 100.

The arrangement of the infrared radiators 110, 112, the mirror elements 130, 132 and the heat sensors 140, 142 shown in FIG. 1 permits the application of heat in the form of heat radiation to a user or a skin area of the user in the irradiation area of the heat treatment apparatus 100. At the same time, it is possible to detect a surface temperature of the skin area by means of the heat sensors 140, 142 during the application of heat radiation by means of the heat treatment apparatus 100 in the skin area. An effect of the heat radiation on the surface temperature in the irradiated skin area may thus be monitored by sensors, for example continuously or at any desired time intervals.

The control means 160 is configured to control the heat output of each of the infrared radiators 110, 112. For this purpose, the control means 160 is operatively connected to the infrared radiators 110, 112. The connection is made, for example, via a dimmer (not shown), which may be controlled by means of the control means 160. In other examples, the control means 160 is configured to output a heating current to each of the infrared radiators 110, 112 that is dimensioned in accordance with an intended heat output.

The control means 160 is further configured to receive sensor signals from each of the heat sensors 140, 142, which indicate a surface temperature detected by the respective sensor 140, 142. A sensor signal from each of the heat sensors 140, 142 indicates a temperature in the detection range of the respective heat sensor 140, 142.

The control means 160 is configured to control a heat output of each of the infrared radiators 110, 112 at least in part based on a surface temperature detected by means of the heat sensors 140, 142 in a skin area of the user which is located in the irradiation area of the heat treatment apparatus 100, in particular to control it depending on the temperature. For this purpose, the processor unit 162 is programmed to compare one or more sensor signals, which are generated by means of one or more of the heat sensors 140, 142 and output to the control means 160 and received by the control means 160, with at least one reference temperature and to vary the heat output of at least one of the infrared radiators 110, 112 as a function of the comparison result. Varying the heat output may correspond to reducing or increasing the heat output.

In some examples of the heat treatment apparatus 100, a functionality of the processor unit 162 is determined by program code stored by means of the memory device 164 and executed by the processor unit 162. In some examples, heat output control is also performed by the control means 160 by comparing a sensed temperature with one or more reference temperatures stored by the memory device 164.

The infrared radiators 110, 112 are controllable in such a way that in an operating state corresponding to a heat output delivered to the user of the heat treatment apparatus 100 in the range of 20 to 120 milliwatts per square centimeter at the skin area of the user, they have a minimum delay of less than 5 seconds to achieve a difference of at least 20% in the heat output delivered to the user in the skin area. Herein, in some examples, a heat source in each of the infrared radiators 110, 112 comprises a ceramic-free heat generating element. A ceramic-free, or in other examples different, heat source in each of the infrared radiators 110, 112 further comprises, for example, a controllable halogen lamp, for example a controllable halogen tube, a controllable infrared, IR, light-emitting diode, LED, and/or a controllable carbon fiber heating element.

The use of infrared radiators 110, 112 as described above enables relatively short variations in the heat output delivered to the user of the heat treatment apparatus 100. In this way, relatively short control spans may be used effectively to control the heat output of the infrared radiators 110, 112 by means of the control means 160. A surface temperature in the irradiated skin area of a user may thus be set more precisely, since over-or under-control may be reduced when regulating the heat output. This reduces the risk of injury or the likelihood of less effective heat treatment as the respective consequences of over-or under-control.

The use of infrared radiators 110, 112 as described above simultaneously allows extended applications of the heat treatment apparatus 100. These include the determination of a heat dissipating capacity of a respective user of the heat treatment apparatus 100. The heat dissipating capacity is determined by the ability of the body to dissipate heat in the skin area to deeper regions of the body. The heat dissipating capacity therefore influences a temperature profile in the irradiated skin area during a heat treatment in relation to a heat output, which is delivered to the user by means of the heat treatment apparatus 100, and an irradiation duration. Knowledge of the user-specific heat dissipating capacity therefore permits improved control of the heat output of the infrared radiators 110, 112 by means of the control means 160 in accordance with an intended temperature. For example, the effect of a variation in heat output on the resulting change in surface temperature in the irradiated area of the user's skin may be estimated in advance on a user-specific basis. Knowledge of a user's heat dissipating capacity may also be used to determine a control span of the control means 160 on a user-specific basis according to a time rate at which the user dissipates applied heat. The efficiency of the control system may thus be improved.

In some examples of the heat treatment apparatus 100, to determine the heat dissipating capacity of a user, the control means 160 is programmed to heat the skin area of the user until a predetermined initial surface temperature is reached and/or for a predetermined period of time to reach a suitable initial surface temperature. The heat treatment apparatus 100 is further programmed to subsequently reduce the heat output of the infrared radiators 110, 112 so that the skin area is cooled rather than heated further. The heat output of the infrared radiators 110, 112 is reduced in such a way that a difference of at least 20% in the heat output delivered to the user in the skin area is achieved in a period of less than 5 seconds. To determine the user's heat dissipating capacity, a surface temperature in the skin area is then recorded, i.e. after the heat output has been reduced, and a temperature profile in the skin area is quantified on the basis of the recorded temperature. The temperature profile is at least partially determined by the user's heat dissipating capacity. For example, by comparing the temperature profile with stored reference profile data using the control means 160, a heat dissipating capacity of the user, for example relative, may be determined. In some examples, a characteristic value for the user's heat dissipating capacity determined in this way is stored in the memory device 164 for later use.

In further examples of the heat treatment apparatus 100, the control means 160 is programmed to increase the heat output of the infrared radiators 110, 112 to determine the heat dissipating capacity of a user, starting from a certain initial surface temperature in the skin area of the user, which corresponds, for example, to an equalized skin temperature at a given room temperature, so that heating of the skin area begins. The heat output of the infrared radiators 110, 112 is increased in such a way that a difference of at least 20% in the heat output emitted to the user in the skin area is achieved in a period of less than 5 seconds. To determine the heat dissipating capacity of the user, a surface temperature in the skin area is then recorded, i.e. after the heat output has been increased and during the continuous delivery of heat output to the user, and a temperature profile in the skin area is quantified on the basis of the recorded temperature. The temperature profile is characterized in particular by an increase in temperature, which is at least partially determined, in particular delayed, by the user's heat dissipating capacity. By comparing the temperature profile with stored reference profile data by means of the control means 160, it is in turn possible to determine, for example, a relative heat dissipating capacity of the user and to store a characteristic value for the heat dissipating capacity of the user determined in this way in the memory device 164 for later use.

The described determination of the heat dissipating capacity is favored by the infrared radiators 110, 112, which permit a relatively short-term variation in the heat output emitted to the user. In this way, a period of time over which the variation in heat output extends is short, and thus a point in time from which the temperature profile may be advantageously quantified may be determined with high accuracy. This allows a relatively accurate calculation of the user-specific heat dissipating capacity in an efficient manner.

The use of the infrared radiators 110, 112 as described above also permits an extension of heat treatments which may be carried out by means of the heat treatment apparatus 100. In particular, it allows heat treatments to be carried out which provide a difference of at least 20% in the heat output delivered to the user in at least one time interval of less than 5 seconds during the course of the heat treatment in question. This makes it possible, for example, to carry out heat stimulation treatments which require short-term variations, i.e. short-term reduction and/or short-term increase, in the heat output delivered to the user in the skin area.

The variation in the heat output delivered to the user in the skin area takes place, for example, with a delay that is less than is required to establish a thermodynamic equilibrium between a heat output delivered to the user at a given time and a surface temperature in the skin area that changes simultaneously as a result of the variation in heat output.

Heat stimulation treatments of the type described may be used to induce or increase the release of adrenalin in the user's body. This is advantageous for so-called activation applications, for example, which have a revitalizing and encouraging effect.

In addition or alternatively, heat stimuli of the type described, for example in the course of a heat treatment, may be used to cause a temporary constriction of the blood vessels through the respective induced or increased release of adrenaline in order to impair heat reduction in the skin area.

This may be used, for example, to increase the effective heat input in a tissue area. For example, a so-called heat depot may be created in the relevant tissue area. Heat depots are suitable for efficiently providing the user with a sustained feeling of warmth over a longer period of time after the heat treatment. Increased effective heat input, as may be achieved by heat stimuli of the type described in the course of a heat treatment, is also advantageous for muscle relaxation applications.

By means of the features described, the heat treatment apparatus 100 also permits reliable control of the heat output on the basis of a predetermined increase in the skin temperature, even with predetermined “steep” increases, i.e. with predetermined increases in the skin temperature in relatively short periods of time.

While a simple, gentle temperature increase in the tissue involves the application of heat over a longer period of time, the use of skin reflex zones to implement physiological effects is based on rapid temperature changes in the skin. The heat treatment apparatus 100 favors various applications in the manner described. Rapid temperature changes require a system that reacts quickly to infrared radiation. Control by a suitable safety system, which may be realized, for example, by suitable regulation, simultaneously prevents accidental thermal damage to the skin.

In some examples of the heat treatment apparatus 100, corresponding program instructions for performing one or more heat treatments of the type described above are stored in the memory device 164.

The use of the infrared radiators 110, 112 as described above also permits improved homogeneity of a heat output distribution in the irradiation area of the heat treatment apparatus 100. Compared to ceramic heat radiators, the infrared radiators 110, 112 comprise, for example, halogen lamps, IR LEDs or carbon fiber elements as heat source, the heat-generating element of which has a relatively small spatial extension. This favors radiation guidance between the respective heat-generating element and the irradiation area, for example by means of reflectors. In the heat treatment apparatus 100 as shown in FIG. 1, radiation is guided, for example, by means of the mirror elements 130, 132. Furthermore, in some examples, the infrared radiators 110, 112 comprise reflectors that increasingly direct the generated infrared radiation in the direction of the mirror elements 130, 132. In addition, in some examples of the heat treatment apparatus 100, an arrangement of one or more radiation-guiding elements is also provided in each of the housing regions 120, 122.

As shown schematically in FIG. 1, in some examples of the heat treatment apparatus 100, the infrared radiators 110, 112 are configured as controllable halogen tubes which are arranged substantially parallel to one another and parallel to the mirror elements 130, 132. The arrangement shown is advantageous when the heat treatment apparatus 100 is used to apply heat to an elongate area of skin, for example along the spine, wherein the heat output should be distributed as evenly as possible over the entire skin area.

The heat treatment apparatus 100 as shown in FIG. 1 also favors a relatively uniform power density of the infrared radiation in a volume above the skin area, for example a volume that extends adjacent to the skin area from a lowest point in the direction of radiation by about 10 cm measured against the direction of radiation. Even if the irradiated skin area is uneven, for example due to curvatures of the spine, it is still possible to apply heat evenly to the entire skin area. The possibility of improved radiation guidance, as made possible by the infrared radiators 110, 112, has a favorable effect.

FIG. 2 schematically and exemplarily shows a heat cabin 200. The heat cabin 200 comprises a cabin wall 210 which surrounds an interior of the heat cabin 200. The heat cabin 200 also comprises a heat treatment apparatus 220 and an overpressure device 230.

The heat treatment apparatus 220 is a heat treatment apparatus as described above in conjunction with the heat treatment apparatus 100 in FIG. 1. To use the heat treatment apparatus 220, a user enters the interior of the heat cabin 210. Compared to a free-standing heat treatment apparatus, the heat cabin 210 favors, for example, heating of the entire body of the user.

The overpressure device 230 is intended to generate an overpressure in the interior of the heat cabin 200. The heat cabin 210 is configured as a pressure chamber for the purpose of hyperbaric therapy applications. The heat cabin 200 is further intended to enable heat treatments by means of the heat treatment apparatus 220 simultaneously with hyperbaric therapy applications.

An overpressure environment has an effect on the operation of the heat treatment apparatus 220 in that, at an increased air pressure, the radiation absorption by the air between the infrared radiators 110, 112 and the skin area to be irradiated is significantly increased. The infrared radiators 110, 112 as described above also favor the provision of infrared radiation in a correspondingly higher heat output range as well as a dynamic adaptation of the heat output to changing pressure conditions in the interior of the heat cabin 210.

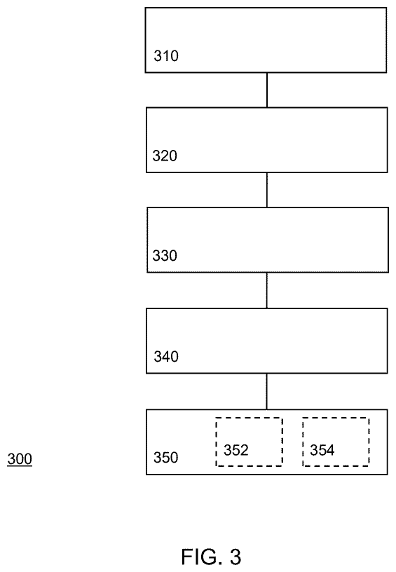

FIG. 3 shows a flowchart of a method 300 for calibrating a heat treatment apparatus according to a user-specific heat dissipating capacity. The heat treatment apparatus is a heat treatment apparatus of the type described above in conjunction with FIGS. 1 and 2.

The method 300 comprises determining an initial surface temperature in the skin area of the user, step 310. After determining the initial surface temperature, the method 300 comprises varying the heat output of the infrared radiator. The variation is carried out in such a way that a difference of at least 20% in the heat output emitted to the user in the skin area is achieved in a period of less than 5 seconds, step 320.

After varying the heat output, the method 300 comprises detecting a surface temperature in the skin region by means of at least one heat sensor, step 330, and determining a temperature profile in the skin region of the user at least in part based on the detected surface temperature by means of the control means, wherein the temperature profile indicates the user's heat dissipating capacity, step 340.

In the method 300, the heat dissipating capacity determined therefrom is also used, for example a characteristic value that indicates the heat dissipating capacity and which is determined from the temperature profile by comparison with a stored reference temperature profile, step 350. The heat dissipating capacity is used to determine a control span for a heat output control of the heat treatment apparatus, sub-step 352, and/or at least one user-related characteristic value that indicates the heat dissipating capacity is stored by means of the memory device, sub-step 354.

The disclosure is described above with reference to specific examples. However, it is understood that some or all of the advantages of the disclosure may also be achieved by implementations of the disclosure which deviate therefrom, for example by using different arrangements or numbers of the described components of a heat treatment apparatus. The numbers and arrangements of the described components may be selected, for example, according to a specific application requirement. It is also understood that advantages of the disclosure may also be achieved in fields of application other than the heat treatment of humans or animals. This includes, for example, the heat treatment of, in particular, thermally reactive materials and/or products.

1. Heat treatment apparatus (100; 220) comprising:
at least one infrared radiator (110, 112) comprising at least one controllable heat source and arranged to emit infrared radiation generated by the heat source to a skin area of a user of the heat treatment apparatus (100; 200);
at least one heat sensor (140, 142) arranged to detect a surface temperature at the skin area of the user; and
a control means (160) adapted to control a heat output of the at least one infrared radiator (110, 112) at least partially based on the detected surface temperature at the skin area of the user,
wherein the infrared radiator (110, 112) has a minimum delay of less than 5 seconds to achieve a difference of at least 20% in the heat output delivered to the user at the skin area, in an operating state corresponding to a heat output delivered to the user in the range of 20 to 120 milliwatts per square centimeter at the skin area of the user.

2. Heat treatment apparatus according to claim 1, wherein the heat source comprises a ceramic-free heat-generating element.

3. Heat treatment apparatus according to claim 1, wherein the heat source comprises a controllable halogen lamp, a controllable infrared, IR, light-emitting diode, LED, and/or a controllable carbon fiber heating element.

4. Heat treatment apparatus according to claim 1, wherein the control means (160) is adapted to control the heat output at least in part as part of a heat output control based on the detected surface temperature.

5. Heat treatment apparatus according to claim 4, wherein the control means (160) is adapted to control the heat treatment apparatus (100; 220) to determine a user-specific heat dissipating capacity at the skin area of the user and, after determining the user-specific heat dissipating capacity, to carry out the heat output control in accordance with a control span that is determined at least in part by the user-specific heat dissipating capacity.

6. Heat treatment apparatus according to claim 5, wherein determining the user-specific heat dissipating capacity comprises:
determining an initial surface temperature in the skin area of the user;
varying a heat output of the infrared radiator (110, 112) so as to effect a difference of at least 20% in the heat output delivered to the user in the skin area of the user within a time period of less than 5 seconds; and
determining a temperature profile at the skin area of the user based at least in part on a surface temperature at the skin area of the user, which is detected by means of the heat sensor (140, 142) after the heat output has been changed, wherein the temperature profile indicates a heat dissipating capacity of the user.

7. Heat treatment apparatus according to claim 6, wherein:
determining the initial surface temperature comprises heating the skin area of the user by use of infrared radiation generated by means of the infrared radiator (110, 112) to attain the initial surface temperature, and varying the heat output comprises decreasing the heat output, wherein the temperature profile corresponds to a cooling of the skin area at least in part due to heat dissipation in the user's body, or
varying the heat output comprises increasing the heat output, wherein the temperature profile corresponds to a heating of the skin area which is at least in part determined by heat dissipation in the user's body.

8. Heat treatment apparatus according to claim 1, wherein the control means (160) includes a memory device (164) configured to store program code for operating the heat treatment apparatus (100; 220) according to at least one heat treatment program, wherein, optionally, the memory device (164) includes program code for operating the heat treatment apparatus (100; 220) according to at least one heat treatment program that provides, during a heat treatment, in at least one time segment of less than 5 seconds, a difference of at least 20% in the heat output delivered to the user at the skin area of the user.

9. Heat treatment apparatus according to claim 1, further comprising at least one mirror element (130, 132) configured to redirect infrared radiation emitted by means of the infrared radiator (110, 112) toward the skin area of the user, wherein, optionally, the heat sensor (140, 142) is arranged in an area of the mirror element (130, 132) and, further optionally, a detection area of the heat sensor (140, 142) extends at least in part in a portion of a radiation cone that is determined by the redirected infrared radiation.

10. Heat treatment apparatus according to claim 9, wherein the heat treatment apparatus (100; 220) comprises at least two mirror elements (130, 132) and at least two infrared radiators (110, 112) arranged on different sides with respect to the mirror elements (130, 132), wherein optionally, the heat source of each of the infrared radiators (110, 112) comprises a controllable halogen tube, wherein the halogen tubes are aligned at least substantially parallel to one another, and wherein a mirror surface of each of the mirror elements (130, 132) is aligned at least substantially parallel to a longitudinal extension direction of at least one of the halogen tubes.

11. Heat treatment apparatus according to claim 1, wherein the heat treatment apparatus (100; 220) comprises a plurality of heat sensors (140, 142) arranged to detect a surface temperature in each of different sub-areas of the skin area of the user, wherein the control means (160) is configured to control the heat output based at least in part on the detected surface temperature in the different sub-areas of the skin area of the user.

12. Heat treatment apparatus according to claim 1, wherein the skin area of the user has a square measure of at least 400 square centimeters and the heat treatment apparatus (100; 220) is configured such that in a volume that extends from the skin area and up to a distance of at least 10 centimeters from at least one point of the skin area parallel to a direction of incidence of the infrared radiation, a location-dependent power density of the infrared radiation at any different points in the volume differs by at most 8%.

13. Heat cabin (200) comprising a heat treatment apparatus (100; 220) according to claim 1.

14. Heat cabin according to claim 13, wherein the heat cabin (200) is further configured as a pressure chamber, in particular for the purpose of hyperbaric therapy applications.

15. Method (300) for user-related calibration of a heat treatment apparatus (100; 220), wherein the heat treatment apparatus (100; 220) comprises:
at least one infrared radiator (110, 112) comprising at least one controllable heat source and arranged to emit infrared radiation generated by means of the heat source to a skin area of a user of the heat treatment apparatus (100; 220);
at least one heat sensor (140, 142) arranged to detect a surface temperature at the skin area of the user; and
a control means (160) configured to control a heat output of the at least one infrared radiator (110, 112) based at least in part on the detected surface temperature at the skin area of the user,
wherein the method (300) comprises the steps of:
determining an initial surface temperature at the skin area of the user;
changing, after determining the initial surface temperature (310) and by use of the control means (160) and the infrared radiator (110, 112), a heat output of the infrared radiator (110, 112) in such a way that in a period of less than 5 seconds a difference of at least 20% in the heat output delivered to the user at the skin area of the user is attained;
detecting (330), after changing the heat output, a surface temperature at the skin area of the user by means of the heat sensor (140, 142);
determining (340), by means of the control means (160) and based at least in part on the detected surface temperature, a temperature profile at the skin area of the user, wherein the temperature profile indicates a user's heat dissipating capacity; and
determining (350, 352), by means of the control means (160) and at least in part based on the user's heat dissipating capacity, a control span for a heat output control of the heat treatment apparatus (100; 220), and/or storing (350, 354), by means of a memory device (164) of the control means (160), at least one user-related characteristic value that indicates the user's heat disspating capacity.
